(19) United States
(12) Patent Application Publication
(10) Pub. No.: US 2010/0030219 A1
(43) Pub. Date: Feb. 4, 2010

(54) ORTHOPEDIC NAVIGATION SYSTEM AND METHOD

(75) Inventors: Alexander Lerner, Karmiel (IL); Alexander Nassonov, Kiryat Shmona (IL); Lev Diamant, Korazim (IL)

Correspondence Address: THE NATH LAW GROUP, 112 South West Street, Alexandria, VA, 22314, US

(73) Assignee: L.R.S. ORTHO LTD., Katzrin (IL)

(21) Appl. No.: 12/543,103

(22) Filed: Aug. 18, 2009

Related U.S. Application Data
(63) Continuation-in-part of application No. 12/308,877, filed on Mar. 25, 2009.

(51) Int. Cl. A61B 17/58; G06K 9/00

(52) U.S. Cl. 606/87; 382/132

(57) ABSTRACT

System and methods for establishing, in combination with an imaging device, a desired orientation of a navigated element having a longitudinal axis, with respect to a bone, the system comprising a jig configured for being directly or indirectly fixed relative to the bone; a positioning head supported by the jig and movable with respect thereto; an angle adjustment member mounted within the positioning head, comprising a passage having a guiding axis, the angle adjustment member being configured for receiving in the passage the navigated element so that the longitudinal axis of the navigated element is aligned with the guiding axis, the angle adjustment member being movable with respect to the positioning head; and an alignment pin having a pin proximal end, a pin distal end and a pin axis, and configured for being received in the passage so that the pin axis is aligned with the guiding axis and so that the pin distal end faces towards the bone, the pin further comprising a light source disposed at its proximal end for projecting an alignment beam along the pin axis in the direction away from the bone for the alignment of the imaging device relative to the guiding axis of the passage for use of the imaging device in establishing the orientation of the guiding axis corresponding to the desired orientation of navigated element.

This is a Continuation-In-Part Application of U.S. patent application Ser. No. 12/308,877 filed on Dec. 29, 2008, which is a National Phase of PCT Application No. PCT/IL2007/000809 filed on Jul. 1, 2007, which claims priority from U.S. Patent Application No. 60/817,102 filed on Jun. 29, 2006, the contents of all of which are hereby incorporated by reference in their entirety.

### FIELD OF THE INVENTION

This invention relates to orthopedic navigation system and method, in particular for establishing a desired orientation of a navigated element.

### BACKGROUND OF THE INVENTION

Systems and methods of the kind to which the subject matter of the present application refers are disclosed, for example, in the following publications: WO 2008/001386 to the Applicant, US 2008/0075348, WO 2004/069063, U.S. Pat. No. 5,411,503 and WO 2003/043485.

### SUMMARY OF THE INVENTION

According to one aspect of the subject matter of the present application, there is provided an orthopedic navigation system for establishing, in combination with an imaging device, a desired orientation of a navigated element having a longitudinal axis, with respect to a bone, the system comprising:
a jig configured for being directly or indirectly fixed relative to the bone;
a positioning head supported by the jig and movable with respect thereto;
an angle adjustment member mounted within the positioning head, comprising a passage having a guiding axis, the angle adjustment member being configured for receiving in the passage the navigated element so that the longitudinal axis of the navigated element is aligned with the guiding axis, the angle adjustment member being movable with respect to the positioning head; and
an alignment pin having a pin proximal end, a pin distal end and a pin axis, and configured for being received in the passage so that the pin axis is aligned with the guiding axis and so that the pin distal end faces towards the bone, the pin further comprising a light source disposed at its proximal end for projecting an alignment beam along the pin axis in the direction away from the bone for the alignment of the imaging device relative to the guiding axis of the passage for use of the imaging device in establishing the orientation of the guiding axis corresponding to the desired orientation of navigated element.

The system can further comprise a reference pin configured for being received in the passage of the angle adjustment member so that the reference pin's axis is aligned with the guiding axis of the passage, the reference pin comprising reference marks visible in images made by the imaging system, in particular being coaxial with the reference pin axis, being disposed at predetermined locations therealong and having predetermined radial dimensions.

The alignment pin can constitute the reference pin.

The imaging device can be a C-arm X-ray machine configured for taking X-ray images of the bone, and at least a portion of the positioning head and/or angle adjustment member associated with the passage, with or without the alignment or reference pin therein.

The C-Arm can comprise a mirror configured for use, together with the collimated light source, for aligning the C-Arm's axis with the alignment pin axis.

The light source of the alignment pin can be configured for projecting the alignment beam along the guiding axis in the direction towards the mirror for obtaining a reflected beam to be aligned with the alignment beam.

The system can further comprise X-ray image processing hardware and software for processing X-ray images of the bone, the alignment/reference pin and at least a portion of the positioning head associated with the passage, for evaluation of positioning parameters determining the displacement of the positioning head or of the angle adjustment member required for bringing the guiding axis to the desired orientation.

The system can further comprise a screen configured for being mounted to the positioning head or to the jig for providing an indication of the displacement of the angle adjustment member relative to the positioning head, required for bringing the guiding axis to the desired orientation.

The navigated element can be selected from the group comprising a drilling bit, a drilling bit guide, a drilling tool including a drill driven by a motor or any other power drive, a biopsy probe, a penetrating guide wire and the like.

According to another aspect of the subject matter of the present application, there is provided orthopedic navigation system for establishing, in combination with an imaging device, a desired orientation of a navigated element having a longitudinal axis, with respect to a bone, the system comprising:
a jig configured for being directly or indirectly fixed relative to the bone;
a positioning head supported by the jig and movable with respect thereto;
an angle adjustment member mounted within the positioning head, comprising a passage having a guiding axis, the angle adjustment member being configured for receiving in the passage the navigated element so that the longitudinal axis of the navigated element is aligned with the guiding axis, the angle adjustment member being movable with respect to the positioning head; and
an alignment pin having a pin proximal end, a pin distal end and a pin axis, and configured for being received in the passage so that the pin axis is aligned with the guiding axis and so that the pin distal end faces towards the bone, the pin further comprising a light source disposed at its proximal end for projecting an alignment beam along the pin axis in the direction away from the bone for the alignment of the imaging device relative to the guiding axis of the passage for use of the imaging device in establishing the orientation of the guiding axis corresponding to the desired orientation of navigated element; and
a screen configured for being mounted to the positioning head or to the jig so as to allow the alignment beam's impingement thereon when the alignment pin is mounted in the passage, for providing an indication of displacement of the angle adjustment member relative to the positioning head.

The system can further comprise means for evaluating positioning parameters determining at least the displacement of the angle adjustment member required for bringing the guiding axis to the desired orientation. The positioning parameters can be distances and/or angles defining the linear displacement of the positioning head or the angular displacement of the angle adjustment member, respectively.

According to another aspect of the subject matter of the present application, there is provided a method for establishing a desired orientation of a navigated element having a longitudinal axis, with respect to a bone, by means of a system comprising:
a jig;
a positioning head supported by the jig and movable with respect thereto;
an angle adjustment member mounted within the positioning head, comprising a passage having a guiding axis, the angle adjustment member being configured for receiving in the passage the navigated element so that the longitudinal axis of the navigated element is aligned with the guiding axis, the angle adjustment member being movable with respect to the positioning head; and
an alignment pin having a pin proximal end, a pin distal end and a pin axis, and configured for being received in the passage so that the pin axis is aligned with the guiding axis and so that the pin distal end faces towards the bone, the pin further comprising a light source disposed at its proximal end for projecting an alignment beam along the pin axis in the direction away from the bone for the alignment of the imaging device relative to the guiding axis of the passage for use of the imaging device in establishing the orientation of the guiding axis corresponding to the desired orientation of navigated element;
   the method comprising:
(a) directly or indirectly fixing the jig relative to the bone;
(b) providing an imaging device and aligning it with respect to the guiding axis by means of the collimated light source and taking images of the bone and at least a portion of the positioning head associated with the passage;
(c) performing displacement of at least one of the angle adjustment member and the positioning head, while a displacement value is estimated based on the images; and
(d) repeating step (c), if necessary, as many times as required to establish the orientation of the guiding axis of the passage corresponding to the desired orientation of the navigated element.

The method can further comprise pre-adjusting the positioning head before the step (a) to bring the guiding axis as close as possible to its desired orientation. At this stage the angle adjustment member is usually adjusted to an initial position in which the angle of the guiding axis relative to the positioning head is zero.

The method may further comprise processing X-ray images of the bone and at least a portion of the positioning head associated with the passage, with hardware and software for evaluation of positioning parameters determining the displacement of the positioning head and/or the angle adjustment member required for bringing the guiding axis to its desired orientation.

The method may further comprise displacing the angle adjustment member together with the alignment pin in accordance with the evaluated positioning parameters with the assistance of a screen configured for being mounted to the positioning head or to the jig for providing an indication of the displacement of the angle adjustment member relative to the positioning head.

The method may further comprise adding a reference object in the proximity of the bone and the field of view of the imaging device to facilitate the evaluation of positioning parameters in step (c).

According to another aspect of the subject matter of the present application, there is provided a method for establishing a desired orientation of a navigated element having a longitudinal axis, by means of a system comprising:
a positioning head movable with respect to the bone;
angle adjustment member mounted within the positioning head, comprising a passage having a guiding axis, the angle adjustment member being configured for receiving in the passage the navigated element so that the longitudinal axis of the navigated element is aligned with the guiding axis;
an alignment pin having a pin proximal end, a pin distal end and a pin axis, and configured for being received in the passage so that the pin axis is aligned with the guiding axis and so that the pin distal end faces towards the bone, the pin further comprising a light source disposed at its proximal end for projecting an alignment beam along the pin axis in the direction away from the bone for the alignment of the imaging device relative to the guiding axis of the passage for use of the imaging device in establishing the orientation of the guiding axis corresponding to the desired orientation of navigated element; and
a screen configured for being mounted to the positioning head or to the jig so as to allow the alignment beam's impingement thereon when the alignment pin is mounted in the passage, for providing an indication of displacement of the angle adjustment member relative to the positioning head;
   the method comprising:
(a) directly or indirectly fixing the jig relative to the bone;
(b) providing an imaging device and aligning it with respect to the guiding axis by means of the collimated light source and taking images of the bone and at least a portion of the positioning head associated with the passage;
(c) evaluating of positioning parameters determining a displacement of at least one of the angle adjustment member and the position head, required for bringing the guiding axis to the desired orientation; and
(d) displacing the angle adjustment member together with the alignment pin in accordance with the evaluated positioning parameters with assistance of the screen.

### BRIEF DESCRIPTION OF THE DRAWINGS

In order to understand the subject matter and to see how it may be carried out in practice, embodiments will now be described, by way of non-limiting example only, with reference to the accompanying drawings, in which:

FIG. 1A is schematic perspective view of a system according to the subject matter of the present application;

FIG. 1B is schematic perspective view of the system shown in FIG. 1A with a navigated element held thereby;

FIG. 2A is a schematic perspective view of the system shown in FIGS. 1A and 1B as used with an alignment pin and a C-arm X-ray machine;

FIGS. 2B and 2C are schematic side and front views, respectively, of one example of a adjustment device for the adjustment of the C-arm shown in FIG. 2A;

FIG. 3 is a schematic illustration of a reference pin to be used with a system of the kind shown in FIGS. 1A to 2A;

FIG. 4 is schematic perspective view of the system shown in FIGS. 1A to 2A as used with the reference pin shown in FIG. 3, having features of the alignment pin shown in FIG. 2A;

FIG. 5 is schematic perspective view of the system shown in FIG. 4 as used with a screen;

FIGS. 6 to 8 are schematic perspective views of a system similar to the system shown in FIGS. 1A to 2A, 4 and 5, used for a variety of orthopedic navigation applications; and

FIGS. 9A to 9C are schematic illustrations of portions of X-ray images taken during the use of the system shown in FIG. 8.

### DETAILED DESCRIPTION OF EMBODIMENTS

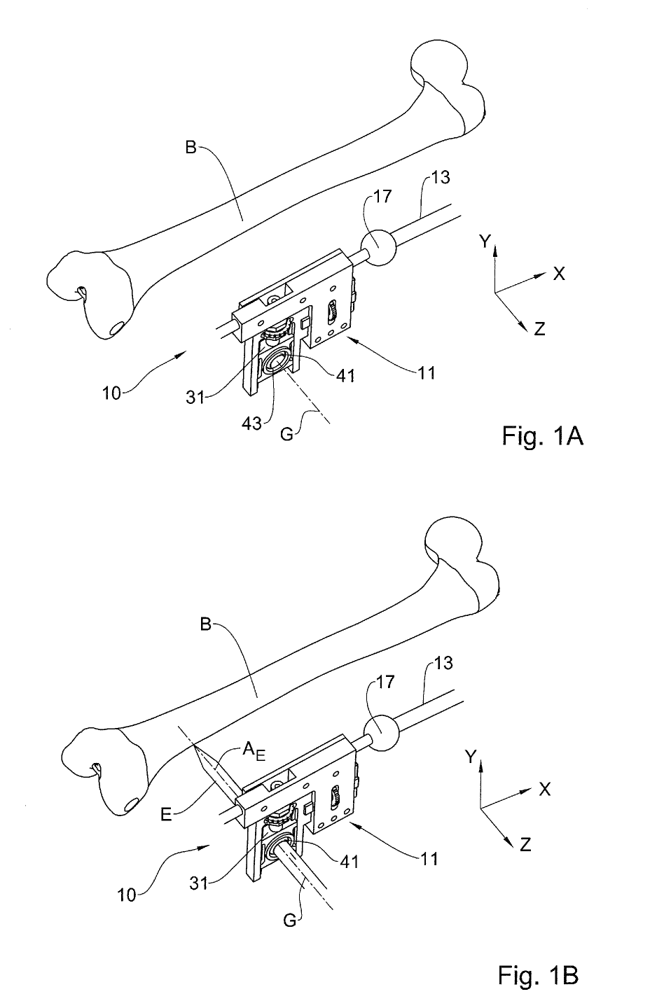

FIGS. 1A and 1B schematically illustrates an orthopedic navigation system 10 for navigating a navigated element E (schematically illustrated in FIG. 1B) that is required to be positioned in a desired orientation with respect to a region of interest in a bone B of a patient during an orthopedic procedure, as will be further explained in detail. The navigated element E may be any suitable working tool such as a drilling bit or a guide for positioning the drill bit, a drilling tool including a drill driven by a motor or any other power drive, a biopsy probe, a guiding wire or the like, corresponding to the procedure it is used for. The bone B is shown in FIG. 1 schematically and without the corresponding body member of the patient, to which the bone B belongs.

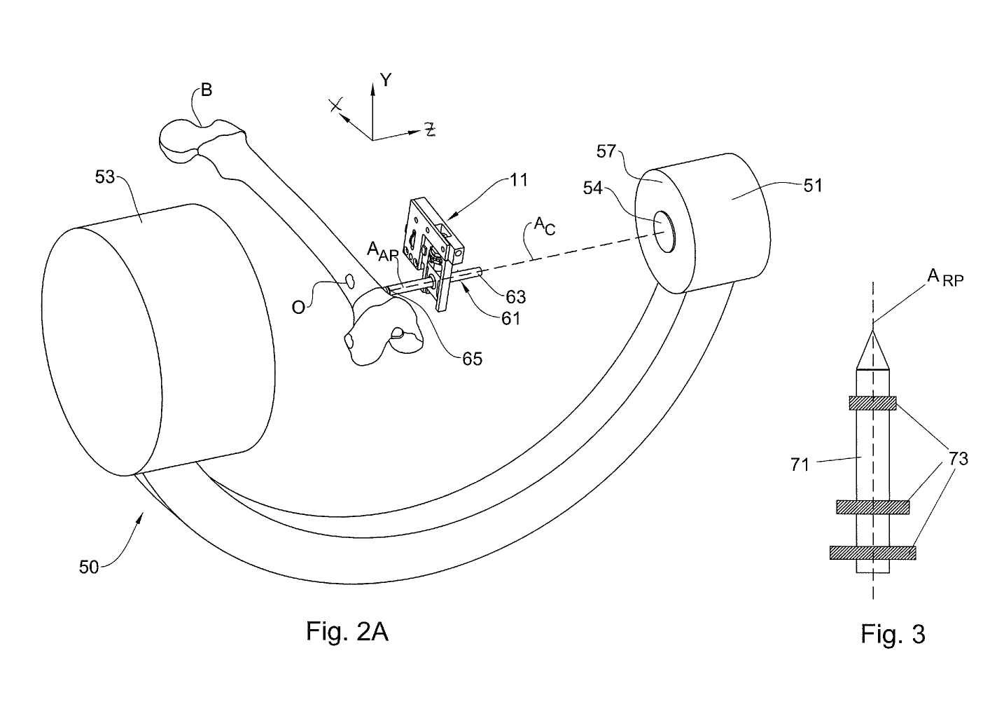

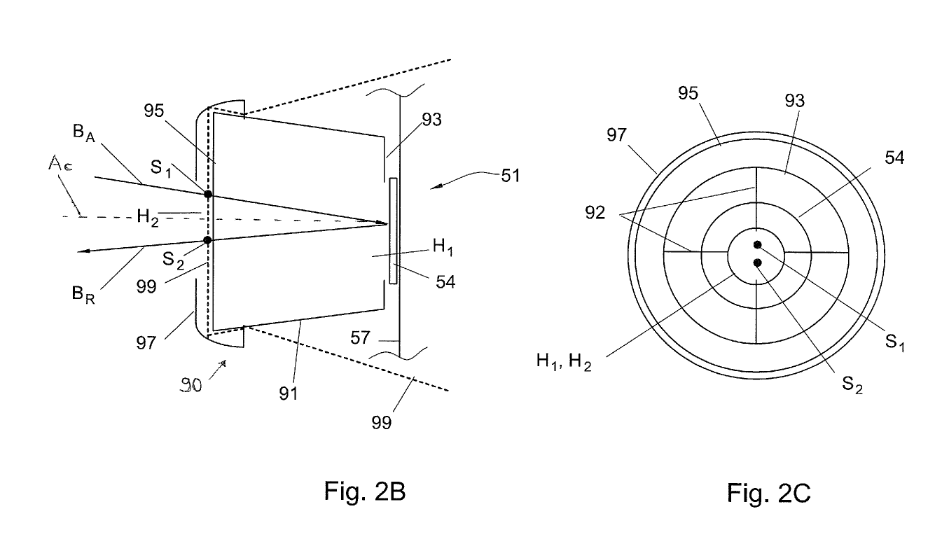

The system 10 comprises a positioning device 11 including a positioning head 31 and an angle adjustment member 41 mounted within the positioning head 31 and configured for receiving therein the navigated element E, and a jig 13 (shown only partially in FIG. 1 and not shown in FIGS. 2, 4 and 5) for supporting the positioning device 11 and fixing it directly or indirectly to the bone B, optionally by means of a supporting arrangement 17, schematically illustrated in FIG. 1A.

With reference to X-Y-Z coordinate system of the positioning device 11 (where the X-Y plane is generally parallel to bone, when the system is mounted thereon, and Z axis is perpendicular thereto), the positioning head 31 comprises positioning screws 31a and 31b allowing linear displacement of the positioning head 31 with respect to the jig 13, and consequently, with respect to the bone B, along X and Y axes, respectively. Each of the positioning screws 31a and 31b can be brought either into a released or a locked position. The released position of the screws 31a and 31b allows the linear displacement of the positioning head 31 as indicated above, while the locked position of the screws 31a and 31b prevents the positioning head 31 from moving along the corresponding axes. As the displacement only along one of the axes may be required, one of the screws can remain in its locked position with the other one being released.

The angle adjustment member 41 is mounted within the positioning head 31 and it comprises a passage 43 having a guiding axis G which is shown in FIG. 1 as extending along Z axis, angle adjustment member. As shown in FIG. 1B, the passage 43 is configured to receive therein the navigated element E, so that an axis A_{E} of the navigated element E is aligned with the guiding axis G of the passage 43. Therefore, bringing the navigated element E to the desired orientation, as indicated above, actually requires bringing the guiding axis G to the desired orientation and then inserting the navigated element E into the passage G to take this orientation.

The angle adjustment member 41 is configured to be displaced with respect to the positioning head 31 so as to change the angular orientation of the guiding axis G relative to the Z axis. The angle adjustment member 41 has a locking screw 45 (not seen) which can be brought into a released position in which the angle adjustment member 41 can be angularly displaced, or in a locked position, preventing such displacement.

The angle adjustment member 41 can be designed as a ball joint or an angular joint in systems where a limited angular displacement is sufficient.

In operation, the system 10 is used together with a C-Arm X-ray machine 50, as shown in FIG. 2A. The C-Arm 50 has an optical axis Ac and comprises an emitter 53 and an X-ray receiver 51 at opposite ends of the axis Ac, and the system 10 is positioned in between the emitter 53 and the receiver 51.

The C-Arm 50 is rotatable with respect to the bone B, so that X-ray images of at least a part of the system, in particular, of at least a part of the positioning head 31 and a region of interest of the bone B may be taken by the C-Arm from different angles.

To simplify the interpretation of the X-ray images and improve its accuracy it is required that the axis A_{C} of the C-Arm 50 be aligned with the guiding axis G of the passage 43 of the angle adjustment member 41. For this purpose, the system 10 further comprises an alignment pin 61 configured to be received within the passage 43 so that its axis A_{AP} is aligned with the guiding axis G of the passage 43 and the C-Arm is provided with a mirror attached to the receiver 51 at the center of its radiation receiving face 57.

The alignment pin 61 has a proximal end 63 and a distal end 65, so that when received within the passage 43, the proximal end 63 faces the receiver 51 of the C-Arm 50 and the distal end 65 faces the emitter 53 of the C-Arm. The alignment pin 61 comprises a collimated light source 67 at its proximal end 63, such as, for example, a laser pointer, for projecting an alignment beam B_{A} (shown in FIG. 2B) along the alignment pin axis A_{AP}, which is aligned with the guiding axis G, towards the mirror 54.

The mirror 54 can constitute a part of an adjustment device configured to position a transparent screen in front of the mirror 54. The adjustment device may include a housing with a proximal end facing the receiver and a distal end facing the bone B. The distal end has an aperture H2 with the screen disposed within the aperture, and the proximal end is configured to provide an optical access to the mirror 54 for a radiation beam entering the housing through the aperture and passes through the screen. With reference to FIG. 2B, the housing may comprise a box 91 having the proximal and distal ends, 93 and 95, respectively, which may be made of plastic and be disposable. The box 91 can be open at its distal end 95, and the screen can be formed by a thin transparent plastic film 99 covering the distal end 95 of the box. The box 91 is provided with a cover 97 having the aperture H₂, covering the distal end of the box, e.g. in a snap-on manner, and holding the film 99 in tension. The proximal end 93 of the box is formed with an opening H₁ aligned with the aperture H₂, the mirror 54 being attached thereto so that its center, as well as the centers of the aperture H₂ and the opening H₂ are disposed on the axis Ac of the receiver 51. The mirror 54 and the cover 95 comprise centering marks 92 as shown in FIG. 2C.

In case that the position of the receiver 51 and the emitter 53 with respect to the bone B is opposite to that shown in FIG. 2, the mirror 54 can be attached to the emitter 53.

With reference to FIG. 3, the system 10 may further comprise a reference pin 71 configured to be received within the passage 43, so that its longitudinal axis A_{RP} is aligned with the guiding axis G. The reference pin 71 comprises reference marks 73 on its outer surface, which are visible in X-ray images. The reference marks 73 may be formed as rings spaced from each other along the axis A_{RP} of the reference pin 71, as shown in FIG. 3. Alternatively, the reference marks 73 may be disposed within the body of the reference pin 71 and/or may have a shape, other than a ring shape, easy recognizable in X-Ray images.

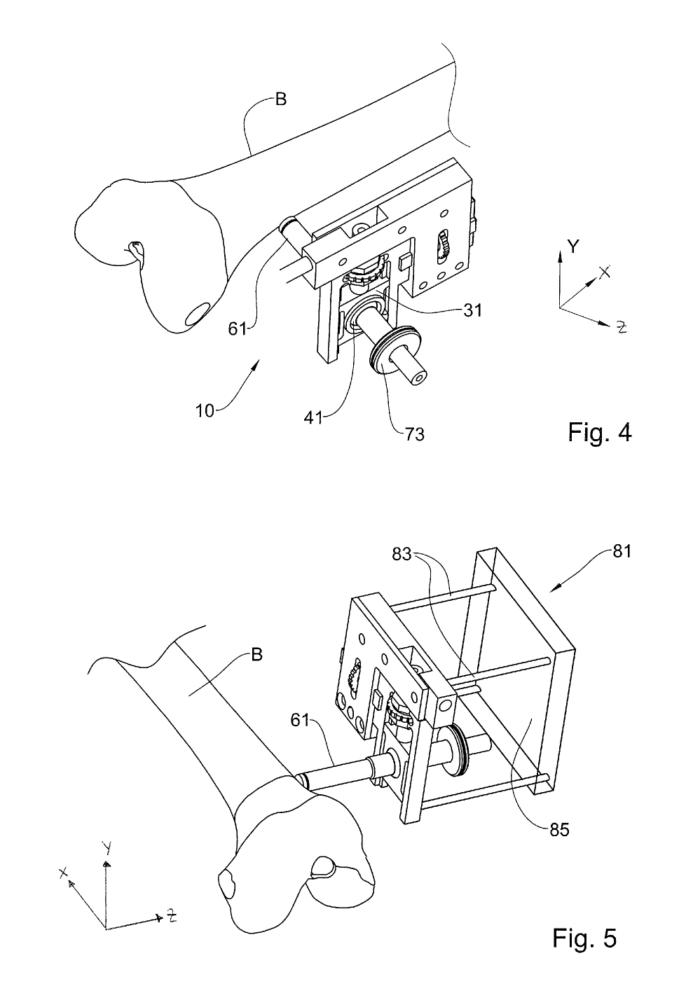

The system 10 can comprise the reference pin 71 as described above, in addition to the alignment pin 61 or, alternatively, the alignment pin 61 may constitute the reference pin 71, as shown in FIG. 4.

The use of the system 10 for establishing the desired orientation of the guiding axis G of the passage 43 and, consequently, of the navigated element E to be received within the passage 43, after the passage is oriented as desired, comprises the following steps:
1. Pre-adjustment of the positioning head 31 prior to the attachment of the jig 13 to the bone B to bring the positioning head as close as possible to the region of interest on the bone B;
2. Initial adjustment of the C-Arm 50 to align its optical axis with the guiding axis G;
3. Taking X-ray images of the region of interest on the bone B and the system 10;
4. Evaluating, based on the X-ray images, the extent to which the positioning head 31 should be displaced and/or the angle adjustment member 41 should be adjusted, to bring the guiding axis G to its desired orientation;
5. Performing displacement(s) of the positioning head 31 and/or angular adjustment of the angle adjustment member, as evaluated in step 4.
6. Adjusting the C-Arm 50 according to the new position of the guiding axis G and taking additional, control X-ray images.
   Each of the above stages will now be described in more detail.
   1. Pre-adjustment of the positioning head 31 prior to the attachment of the jig 13 to the bone B to bring the positioning head as close as possible to the region of interest on the bone B.

The purpose of this stage is to bring the positioning head 31 together with the angle adjustment member 41 to a position as accurate as possible relative to the bone B, in particular to the region of interest to which the navigated element E should later be brought, thereby preventing the necessity in large displacements of the positioning head 31 during the operation. The pre-adjustment is performed based on preliminary information available before the medical procedure, such as CT scan of the bone B and/or other computerized data, dimensions of known, reference objects which can be, parts of the system 10, the position of an entry point on the surface of the bone, etc.

One or both of the positioning screws 31a and 31b are released and the positioning head 31 is displaced along X and/or Y axes, respectively, to the desired position, and then locked with respect to the jig 13, so that when the jig 13 is mounted to the bone B the positioning head 31 remains in the pre-adjusted position.

At this stage the angle adjustment member 41 is in its initial position in which the guiding axis G is parallel to the Z axis, i.e. its angle with respect to the Z axis equals zero (hereinafter a “zero” position of the guiding axis G).

2. Initial adjustment of the C-Arm 50 to align its optical axis with the guiding axis G.

The jig 13 is attached to the bone B directly or by means of other elements, as will be further detailed, while the positioning head 31 is in its pre-adjusted position. The alignment pin 61 is then inserted into the passage 43 of the angle adjustment member 41 and the collimated light source 67 projects the alignment beam B_{A}, which is aligned with the guiding axis G of the passage 43, towards the center of the mirror 54 on the receiver 51 of the C-Arm, so as to obtain a beam B_{R} (shown in FIG. 2B) reflected from the mirror 54. The coincidence of such reflected beam B_{R} with the alignment beam B_{A} will indicate that the axis of the C-Arm 50 is aligned with the guiding axis G. This coincidence is achieved by watching a spot created by the reflected beam B_{R} on a plane surface, e.g. a sheet of paper, positioned between the collimated light source 67 and the receiver 51, and adjusting the C-Arm 50 so that the spot is located exactly on the light source 67.

Alternatively, the adjustment of the C-Arm 50 can be achieved with the assistance of the adjustment device 90, as shown in FIG. 2B. The collimated light source 67 projects the alignment beam BA towards the center of the mirror 54, indicated by the centering marks 92 produces a first visible scattered spot S₁ on the film 99 (FIG. 2C). The reflected beam B_{R} produces a second visible scattered spot S₂ on the film. The coincidence of the spots S₁ and S₂ indicate that the alignment beam B_{A} is aligned with the reflected beam B_{R}, i.e. the axis A_{C} of the C-Arm 50 is aligned with the guiding axis G.

3. Taking X-ray images of the region of interest on the bone B and the system 10.

Once the C-Arm 50 is adjusted, several (usually 2-3) X-ray images of the system 10 with the bone B are taken at orthogonal angles.

At this stage, it is desired to have at least one reference object within the field of view of the C-Arm 50 for facilitating the analysis of the images. For this purpose, the alignment pin 61 can be replaced with the reference pin 71, so that the images include at least the reference pin with the reference marks 73 visible in the images and the bone B.

Alternatively or additionally, a reference object O (schematically shown in FIG. 2) can be added within the field of view of the C-Arm 50, for example within the bone or in the proximity thereto. The X-ray images will then include the reference object O in addition to the system 10 and the bone B. The reference object O has to be of a known shape and dimensions, visible on X-ray images, easily insertable in and extractable from the field of view of the C-Arm and making no harm to the bone.

The reference marks 73 and/or the reference object O allow to compare different parameters as seen in the X-ray images, i.e. distances and angles between different parts of the system and the bone B, with their real values.

4. Evaluating, based on the X-ray images, the extent to which the positioning head 31 should be displaced and/or the angle adjustment member 41 should be adjusted, to bring the guiding axis G to its desired orientation.

The surgeon imagines in his mind a 3-D image of the region of interest of the bone B based on the X-ray images taken in the previous stage of the procedure and on the information he obtained before the pre-adjustment stage and estimates the extent and direction in which the positioning head 31 and/or the angle adjustment member 41 has to be displaced, if at all, to bring the guiding axis G as close as possible to its desired orientation.

5. Performing displacement(s) of the positioning head 31 and/or angular adjustment of the angle adjustment member, as evaluated in step 4.

One or both positioning screws 31a and 31b are released and the positioning head 31 is displaced along X and/or Y axes according to the displacement values estimated in step 4. The screws 31a and/or 31b are then locked again.

Once the positioning head 31 has been displaced as described above and locked in such position, the angle adjustment member 41 is adjusted relative to Z axis to bring the guiding axis G to its desired orientation, as estimated according to the X-ray images and the information available before the pre-adjustment stage.

The locking screw 45 of the angle adjustment member 41 is released and the above adjustment is performed. Once the angle adjusted element 41 is displaced to the desired orientation, the locking screw 45 is locked.

The angular adjustment of the angle positioning member 41 can take place before the displacement of the positioning head 31 and both the displacement and the adjustment may be followed and or preceded by the adjustment of the C-Arm, so that its axis A_{C} is aligned with the guiding axis G, as detailed in step 6 below. In addition, control X-ray images can be taken when necessary.

It should be noted, that one or both the displacement of the positioning head 31 and the angular adjustment of the angle positioning member 41 are optional, as no linear or angular adjustment may be required.

6. Adjusting the C-Arm 50 according to the new position of the guiding axis G and taking additional, control X-ray images.

The C-Arm 50 is adjusted based on the updated position of the guiding axis G of the passage 43 using the collimated light source 67 of the alignment pin 61, by an adjustment procedure similar to that performed as explained in stage 2. One or more additional X-ray image is then taken to assure that the guiding axis G is at the desired orientation.

Any of the above stages may be repeated if the surgeon is not satisfied with the results and he feels that an additional linear and/or angular adjustment is needed.

The evaluation of the extent to which the positioning head 31 and the angle adjustment member 41 should be displaced may be replaced by calculations of corresponding positioning parameters by suitable of a computer, i.e. image processing software and hardware. In such a case, the stage 3 above is followed by an additional stage, where the positioning parameters such as linear distances for the displacement of the positioning head 31 along X and/or Y axes and angles for the angular displacement of the angle adjustment member 41 are calculated based on the taken X-ray images and other information, if available.

To facilitate the angular displacement control of the angle adjustment member 41, the system 10 may further comprise a screen 81, configured to be mounted to the positioning head 31 or to a jig 13. With reference to FIG. 4, the screen 81 is optionally made of plastic, has a flat surface 85 comprising angle scale marks thereon, and is mounted to the positioning head 31, e.g. by means of rods 83, so that the flat surface 85 faces the positioning head 31.

In operation, once the angle to which the angle adjustment member 41 has to move the guiding axis G is calculated, the pin 61 with the collimated light source 67 is inserted into the passage 43 of the angle adjustment member 41, and a spot obtained by means of its projection on the screen 81, serves to control angular displacement of the alignment pin 61. As the axis A_{AP} of the alignment pin 61 is aligned with the guiding axis G of the passage 43, the screen 81 allows to bring the guiding axis G to the desired orientation.

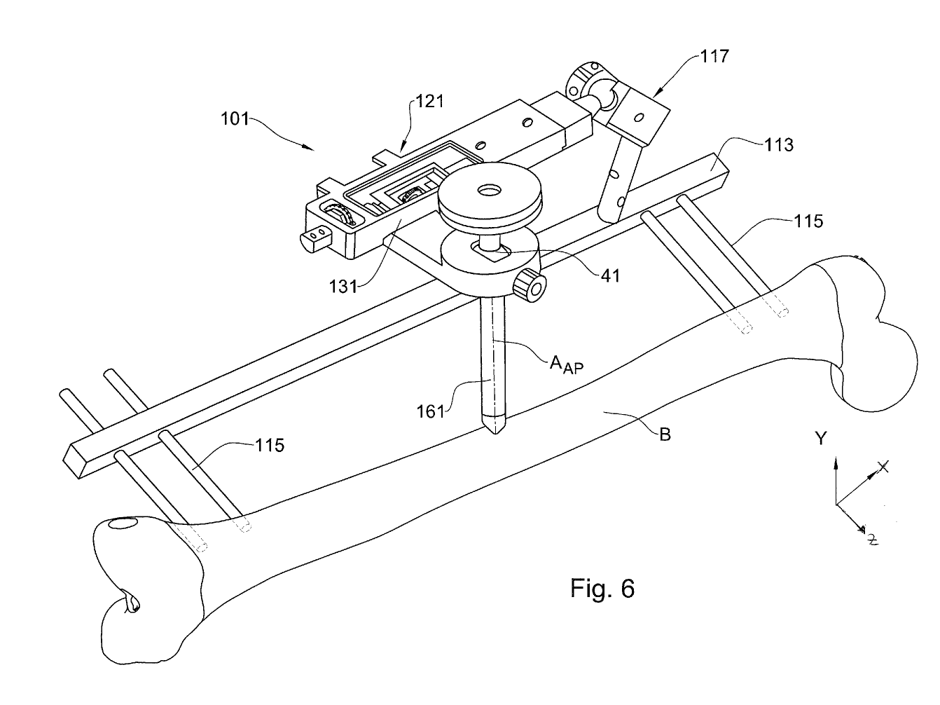

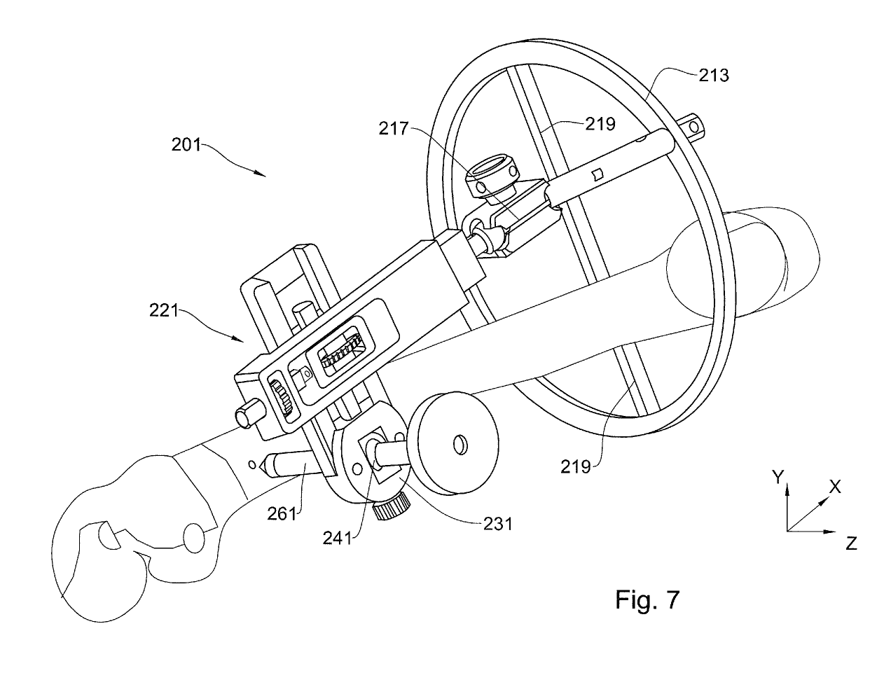

The system 10 can be used for a variety of orthopedic navigation applications, as described with reference to FIGS. 6 to 8.

With reference to FIG. 6, there is shown a system 101 for bone drilling procedures for purposes such as Osteoid osteoma, bone biopsy or removing foreign substances lodged in the bone.

The system 101 comprises elements similar to those of the system 10, namely a positioning device 121 comprising a positioning head 131, an angle adjustment member 141 mounted within the positioning head 131, an alignment pin 161 and a jig 113. The angle adjustment member 141 is configured for receiving therein the navigated element E, which, in this particular example, is a drilling bit or a drill bit guide, a desired orientation of which is determined by the entry point on the bone B, the anatomy of the region of interest adjacent the entry point, the purpose of the drilling, etc.

The jig 113 is an elongated element mounted to the bone B by means of mounting rods 115 which are substantially parallel thereto. The jig 113 is attached to the positioning device 121 by means of a supporting arrangement 117.

When the system 101 is in use, the jig 113 together with the supporting arrangement 117 creates a rigid bridge between the bone B and the positioning device 121, which facilitates the displacement of the positioning head 131 with respect to the bone B and the use of the navigated element E, for example, during the drilling procedure, by the limitation of the relative movement between the bone B and the positioning device 121.

With reference to FIG. 7, there is shown a system 201 that can be used for osteotomy procedures, such as for example, Ilizarov surgery, where the navigated element E is a cutting tool.

The system 201 comprises elements similar to those of the system 10, namely a positioning device 221 having a positioning head 231 and an angle adjustment member 241 mounted within the positioning head 231, an alignment pin 261 and a jig 213. The angle adjustment member 241 is configured for receiving therein the navigated element E, a desired orientation of which is determined by the region to be cut on the bone B, the anatomy of the region of interest adjacent the region to be cut, the way of cutting, etc.

The jig 213, to which the positioning device 221 is attached by means of a supporting arrangement 217, is a circular fixator, which is fixed to the bone B by means of pins 219.

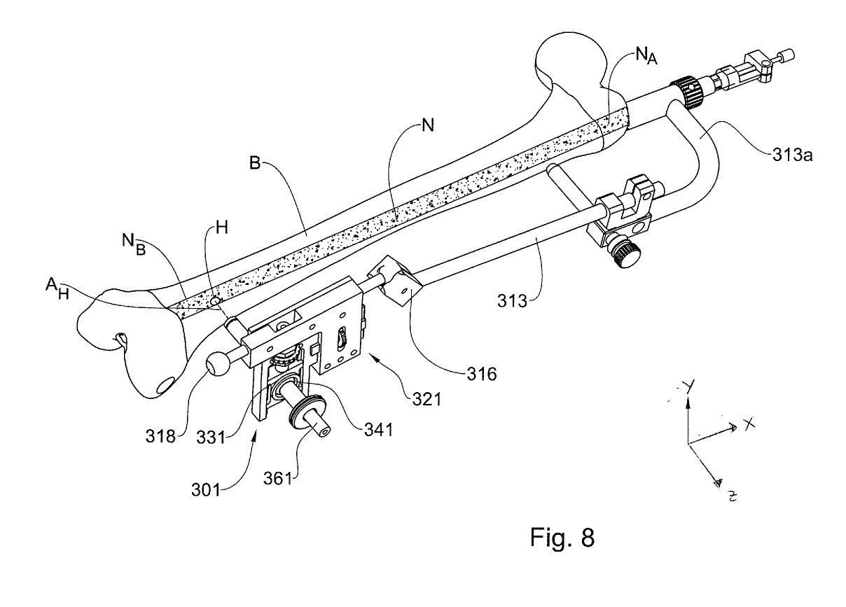

With reference to FIG. 8, there is illustrated a system 301 intended to be used with an intramedullary nail N when implanted into the bone B. The nail has a proximal end N_{A}, a distal end N_{B} and two transverse distal holes H formed in the nail walls adjacent the distal end N_{B}, each having a hole axis A_{H}. The system 301 is configured for determining the orientation of the axes A_{H} of the holes H for further aligning of the navigated element E therewith. The navigated element E in this example is a drilling bit and it is intended for drilling in the bone holes in alignment with the holes H.

The system 301 comprises elements similar to those of the system 10, namely a positioning device 321 having a positioning head 331 and an angle adjustment member 341 mounted within the positioning head 331, an alignment pin 361 and a jig 313.

The jig 313 is configured for being attached at its proximal end 313a to the proximal end N_{A} of the nail N by a handle 314, with both the proximal and distal support portions 316 and 318 (shown only partly) being attached to the bone B.

In operation, the jig 313 together with the handle 314 creates a rigid bridge between a distal end of the bone B and the proximal end N_{A} of the nail N, which facilitates a procedure of positioning of the positioning head 331 and then drilling the holes, by the limitation of the movement of the nail N with respect to the bone B. The proximal support portion 316 and the distal support portion 318 of the jig 313 contribute to the stability of the created bridge.

The method of operation of the system 301 is described in co-pending U.S. patent application Ser. No. 12/308,877, first published as WO 2008/001386, whose contents are incorporated herein by reference in their entirety. Briefly speaking, the method described in the above application comprises the steps 1 to 6 detailed above, and certain additional steps including the use of a computer for calculating the positioning parameters, which are optional. On the other hand, U.S. patent application Ser. No. 12/308,877, does not include certain steps and/components of the system suggested in the present application, which can be used with the system 301. For example, the screen 81 can clearly be used with the system 301 to eliminate the need of touching the bone by the alignment pin as suggested in U.S. patent application Ser. No. 12/308,877, as the screen 81 provides a clear indication of the angular movement of the angle adjustment member, which in that application is in the form of a ball joint.

An alternative manner of operation of the system 301, without the use of the computer, will now be described with reference to the stages detailed above in connection with the system 10.

With reference to stage 3, the X-ray images that are taken in this stage include the reference pin 71 with the reference marks 73, at least a part of the positioning head 31 and at least one hole H within the nail N which is implanted within the bone B. The parameters of the hole H are known and do not change after the nail N is implanted within the bone B. However, during the implantation a twisting and/or bending of the nail N may occur. In such a case, the hole and the reference marks as seen in the X-ray images provide the indication to the kind of the distortion which occurred during the implantation of the nail N.

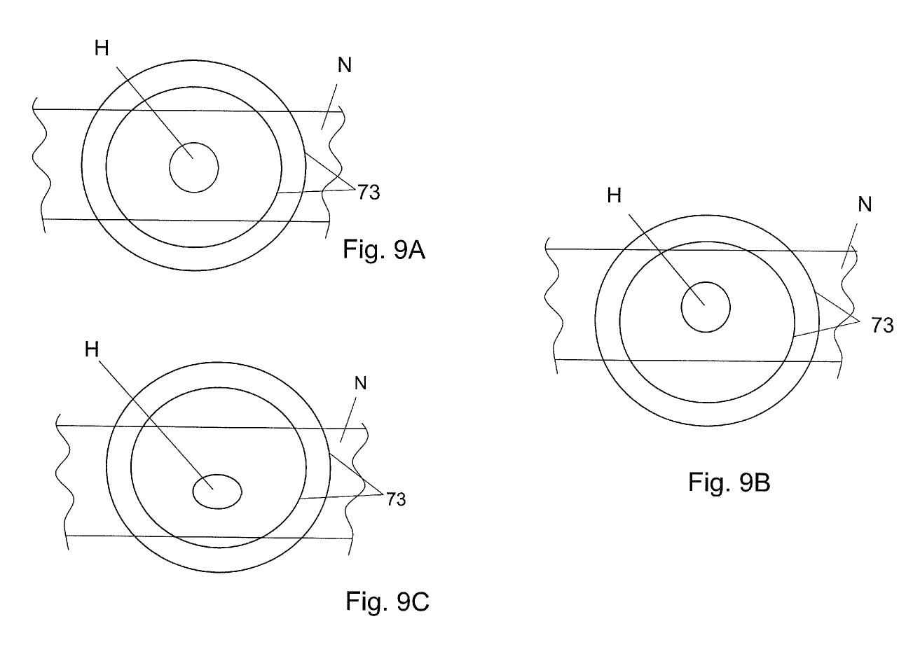

FIGS. 9A to 9C show examples of three possible kinds of X-ray images that can be obtained. As shown in FIG. 9A, the hole H is seen in the image as being circular and concentric with the reference marks 73. In this case it can be concluded that no twisting or bending of the nail N occurred during the implantation and therefore no linear adjustment of the positioning head 331 or angular adjustment of the angle adjustment member 341 are necessary, as the guiding axis G is already in its desired orientation as it was adjusted at stage 1, before the nail N was implanted within the bone B. The drilling bit is then inserted into the passage 343 of the adjustment element 341 and the surgeon proceeds with the drilling procedure.

With reference to FIG. 9B, the hole H is seen in the image as being circular, but not concentric with the reference marks 73, which indicates that the bending of the nail with respect to the X axis occurred during the implantation. Consequently, the hole H was linearly displaced along the Y axis. Therefore, only the displacement of the positioning head 331 along the Y axis is required. The surgeon estimates, based on the X-ray images the value of the displacement, i.e. the distance to which the positioning head 331 has to be displaced along the Y-axis according to stage 4 above. As no twisting of the nail N occurred, no angular displacement of the angle adjustment member 341 is required.

With reference to FIG. 9C, the hole H is seen in the image as being non-circular and non-concentric with the reference marks 73, which indicates that both the bending and the twisting of the nail occurred during the implantation. Consequently, the hole H was linearly displaced along the X axis upwards (as shown in FIG. 9C) or downwards (not shown) and it was also angularly displaced with respect to its original position. Therefore, both the displacement of the positioning head 331 along the X axis and the angular displacement of the angular adjustment element 341 are required.

First, the non-circularity of the hole H has to be fixed. For this purpose, the surgeon has to angularly adjust the C-Arm so that the hole H appears circular on the image, which usually is achieved after about 2 to 3 shots. The angle adjustment member 341 then has to be adjusted according to the new position of the C-Arm, by means of the alignment pin 61 with the collimated light source 67, as described above. Once the hole H is circular and the guiding axis G of the angle adjustment member 341 is aligned with the axis A_{C} of the C-Arm, the surgeon has only to deal with the non-concentricity of the reference marks 73 by the displacement of the positioning head 331, as described with reference to FIG. 9B.

Those skilled in the art to which the subject matter of the present application pertains will readily appreciate that numerous changes, variations, and modifications can be made without departing from the scope of the subject matter, mutatis mutandis. For example, the subject matter of the present application is applicable for orthopedic use with bones different from that shown in the drawings, in procedures and systems different from those disclosed in the application, with the components of the system having design different from that shown and described as long as they perform the same functions as described.

1. An orthopedic navigation system for establishing, in combination with an imaging device, a desired orientation of a navigated element having a longitudinal axis, with respect to a bone, the system comprising:
a jig configured for being directly or indirectly fixed relative to the bone;
a positioning head supported by the jig and movable with respect thereto;
an angle adjustment member mounted within the positioning head, comprising a passage having a guiding axis, the angle adjustment member being configured for receiving in the passage the navigated element so that the longitudinal axis of the navigated element is aligned with the guiding axis, the angle adjustment member being movable with respect to the positioning head; and
an alignment pin having a pin proximal end, a pin distal end and a pin axis, and configured for being received in the passage so that the pin axis is aligned with the guiding axis and so that the pin distal end faces towards the bone, the pin further comprising a light source disposed at its proximal end for projecting an alignment beam along the pin axis in the direction away from the bone for the alignment of the imaging device relative to the guiding axis of the passage for use of the imaging device in establishing the orientation of the guiding axis corresponding to the desired orientation of navigated element.

2. An orthopedic navigation system according to claim 1, further comprising a reference pin configured for being received in the passage of the ball joint so that a reference pin axis is aligned with the guiding axis of the passage, the reference pin comprising reference marks visible in X-ray images, in particular being coaxial with the reference pin axis, being disposed at predetermined locations therealong and having predetermined radial dimensions.

3. An orthopedic navigation system according to claim 2, wherein the alignment pin constitutes the reference pin.

4. An orthopedic navigation system according to claim 2, wherein the imaging device is a C-arm X-ray machine configured for taking X-ray of the bone, and at least a portion of the positioning head and/or angle adjustment member associated with the passage, with or without the alignment or reference pin therein.

5. An orthopedic navigation system according to claim 4, wherein the C-Arm comprises a mirror configured for use, together with the light source, for aligning the C-Arm's axis with the alignment pin axis.

6. An orthopedic navigation system according to claim 5, wherein the light source is configured for projecting the alignment beam along the guiding axis in the direction towards the mirror for obtaining a reflected beam to be aligned with the alignment beam.

7. An orthopedic navigation system according to claim 2, further comprising X-ray image processing hardware and software for processing X-ray images of the bone, the alignment/reference pin and at least a portion of the positioning head associated with the passage, for evaluation of positioning parameters determining the displacement of the positioning head or of the angle adjustment member required for bringing the guiding axis to the desired orientation.

8. An orthopedic navigation system according to claim 1, further comprising a screen configured for being mounted to the positioning head or to the jig for providing an indication of the displacement of the angle adjustment member relative to the positioning head, required for bringing the guiding axis to the desired orientation.

9. An orthopedic navigation system according to claim 1, wherein the navigated element is selected from the group comprising a drilling bit, a drilling bit guide, a drilling tool including a drill driven by a motor or any other power drive, a biopsy probe and a penetrating guide wire.

10. An orthopedic navigation system for establishing, in combination with an imaging device, a desired orientation of a navigated element having a longitudinal axis, with respect to a bone, the system comprising:
a jig configured for being directly or indirectly fixed relative to the bone;
a positioning head supported by the jig and movable with respect thereto;
an angle adjustment member mounted within the positioning head, comprising a passage having a guiding axis, the angle adjustment member being configured for receiving in the passage the navigated element so that the longitudinal axis of the navigated element is aligned with the guiding axis, the angle adjustment member being movable with respect to the positioning head; and
an alignment pin having a pin proximal end, a pin distal end and a pin axis, and configured for being received in the passage so that the pin axis is aligned with the guiding axis and so that the pin distal end faces towards the bone, the pin further comprising a light source disposed at its proximal end for projecting an alignment beam along the pin axis in the direction away from the bone for the alignment of the imaging device relative to the guiding axis of the passage for use of the imaging device in establishing the orientation of the guiding axis corresponding to the desired orientation of navigated element; and
a screen configured for being mounted to the positioning head or to the jig so as to allow the alignment beam's impingement thereon when the alignment pin is mounted in the passage, for providing an indication of displacement of the angle adjustment member relative to the positioning head.

11. An orthopedic navigation system according to claim 10, further comprising means for evaluating positioning parameters determining at least the displacement of the angle adjustment member required for bringing the guiding axis to the desired orientation.

12. An orthopedic navigation system according to claim 10, wherein the positioning parameters are distances.

13. An orthopedic navigation system according to claim 10, wherein the positioning parameters are angles.

14. A method for establishing a desired orientation of a navigated element having a longitudinal axis with respect to a bone, by means of a system comprising:
a positioning head movable with respect to the bone;
angle adjustment member mounted within the positioning head, comprising a passage having a guiding axis, the angle adjustment member being configured for receiving in the passage the navigated element so that the longitudinal axis of the navigated element is aligned with the guiding axis; and
an alignment pin having a pin proximal end, a pin distal end and a pin axis, and configured for being received in the passage so that the pin axis is aligned with the guiding axis and so that the pin distal end faces towards the bone, the pin further comprising a light source disposed at its proximal end for projecting an alignment beam along the pin axis in the direction away from the bone for the alignment of the imaging device relative to the guiding axis of the passage for use of the imaging device in establishing the orientation of the guiding axis corresponding to the desired orientation of navigated element;
the method comprising:
(a) directly or indirectly fixing the jig relative to the bone;
(b) providing an imaging device and aligning it with respect to the guiding axis by means of the collimated light source and taking images of the bone and at least a portion of the positioning head associated with the passage;
(c) performing displacement of at least one of the angle adjustment member and the positioning head, while a displacement value is estimated based on the images; and
(d) repeating step (c), if necessary, as many times as required to establish the orientation of the guiding axis of the passage corresponding to the desired orientation of the navigated element.

15. A method according to claim 14, further comprising pre-adjusting the positioning head before the step (a) to bring the guiding axis as close as possible to its desired orientation.

16. A method according to claim 14, wherein the system further comprises a reference pin configured for being received in the passage of the ball joint so that a reference pin axis is aligned with the guiding axis of the passage, the reference pin comprising reference marks visible in X-ray images, in particular being coaxial with the reference pin axis, being disposed at predetermined locations therealong and having predetermined radial dimensions

17. A method according to claim 16, wherein the alignment pin constitutes the reference pin.

18. A method according to claim 16, wherein the imaging device is a C-arm X-ray machine configured for taking X-ray of the bone, and at least a portion of the positioning head and/or angle adjustment member associated with the passage, with or without the alignment or reference pin therein.

19. A method according to claim 18, wherein the C-Arm comprises a mirror configured for use, together with the light source, for aligning the C-Arm's axis with the alignment pin axis.

20. A method according to claim 19, wherein the light source is configured for projecting the alignment beam along the guiding axis in the direction towards the mirror for obtaining a reflected beam to be aligned with the alignment beam.

21. A method according to claim 14, further comprising processing X-ray images of the bone and at least a portion of the positioning head associated with the passage, with hardware and software for evaluation of positioning parameters determining the displacement of the positioning head and/or the angle adjustment member required for bringing the guiding axis to its desired orientation.

22. A method according to claim 21, further comprising displacing the angle adjustment member together with the alignment pin in accordance with the evaluated positioning parameters with the assistance of a screen configured for being mounted to the positioning head or to the jig for providing an indication of the displacement of the angle adjustment member relative to the positioning head.

23. A method according to claim 14, further comprising adding a reference object in the proximity of the bone and the field of view of the imaging device to facilitate the evaluation of positioning parameters in step (c).

24. A method for establishing a desired orientation of a navigated element having a longitudinal axis, by means of a system comprising:
a positioning head movable with respect to the bone;
angle adjustment member mounted within the positioning head, comprising a passage having a guiding axis, the angle adjustment member being configured for receiving in the passage the navigated element so that the longitudinal axis of the navigated element is aligned with the guiding axis;
an alignment pin having a pin proximal end, a pin distal end and a pin axis, and configured for being received in the passage so that the pin axis is aligned with the guiding axis and so that the pin distal end faces towards the bone, the pin further comprising a light source disposed at its proximal end for projecting an alignment beam along the pin axis in the direction away from the bone for the alignment of the imaging device relative to the guiding axis of the passage for use of the imaging device in establishing the orientation of the guiding axis corresponding to the desired orientation of navigated element; and
a screen configured for being mounted to the positioning head or to the jig so as to allow the alignment beam's impingement thereon when the alignment pin is mounted in the passage, for providing an indication of displacement of the angle adjustment member relative to the positioning head;
the method comprising:
(a) directly or indirectly fixing the jig relative to the bone;
(b) providing an imaging device and aligning it with respect to the guiding axis by means of the collimated light source and taking images of the bone and at least a portion of the positioning head associated with the passage;
(c) evaluating of positioning parameters determining a displacement of at least one of the angle adjustment member and the position head, required for bringing the guiding axis to the desired orientation; and
(d) displacing the angle adjustment member together with the alignment pin in accordance with the evaluated positioning parameters with assistance of the screen.
